SEQUENCE LISTING (50 sequences)

SEQ ID NO: 1 - protein, 361 aa, Homo sapiens
MVVMAPRTLFLLLSGALTLTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 2 - protein, 361 aa, Homo sapiens
MAVMAPRTLLLLLSGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 3 - protein, 361 aa, Homo sapiens
MAVMAPRTLVLLLSGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 4 - protein, 361 aa, Homo sapiens
MAVMAPRTLLLLLLGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 5 - protein, 361 aa, Homo sapiens
MAIMAPRTLVLLLSGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 6 - protein, 361 aa, Homo sapiens
MLVMAPRTVLLLLSAALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 7 - protein, 361 aa, Homo sapiens
MRVTAPRTVLLLLSGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 8 - protein, 361 aa, Homo sapiens
MRVTAPRTVLLLLSAALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 9 - protein, 361 aa, Homo sapiens
MRVTAPRTLLLLLWGAVALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 10 - protein, 361 aa, Homo sapiens
MRVTAPRTLLLLLWGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 11 - protein, 361 aa, Homo sapiens
MRVTAPRTVLLLLWGAVALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 12 - protein, 361 aa, Homo sapiens
MRVMAPRTLILLLSGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 13 - protein, 361 aa, Homo sapiens
MRVMAPRALLLLLSGGLALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 14 - protein, 361 aa, Homo sapiens
MRVMAPRTLLLLLSGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 15 - protein, 361 aa, Homo sapiens
MRVMAPQALLLLLSGALALIETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 16 - protein, 361 aa, Homo sapiens
MVVMAPRTLFLLLSGALTLTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 17 - protein, 361 aa, Homo sapiens
MAVMAPRTLLLLLSGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 18 - protein, 361 aa, Homo sapiens
MAVMAPRTLVLLLSGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 19 - protein, 361 aa, Homo sapiens
MAVMAPRTLLLLLLGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 20 - protein, 361 aa, Homo sapiens
MAIMAPRTLVLLLSGALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 21 - protein, 361 aa, Homo sapiens
MLVMAPRTVLLLLSAALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 22 - protein, 361 aa, Homo sapiens
MRVTAPRTVLLLLSGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 23 - protein, 361 aa, Homo sapiens
MRVTAPRTVLLLLSAALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 24 - protein, 361 aa, Homo sapiens
MRVTAPRTLLLLLWGAVALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 25 - protein, 361 aa, Homo sapiens
MRVTAPRTLLLLLWGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 26 - protein, 361 aa, Homo sapiens
MRVTAPRTVLLLLWGAVALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 27 - protein, 361 aa, Homo sapiens
MRVMAPRTLILLLSGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 28 - protein, 361 aa, Homo sapiens
MRVMAPRALLLLLSGGLALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 29 - protein, 361 aa, Homo sapiens
MRVMAPRTLLLLLSGALALTETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 30 - protein, 361 aa, Homo sapiens
MRVMAPQALLLLLSGALALIETWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 31 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 32 - protein, 358 aa, Homo sapiens [sequence omitted]

SEQ ID NO: 33 - protein, 24 aa, Homo sapiens
MVVMAPRTLFLLLSGALTLTETWA

SEQ ID NO: 34 - protein, 24 aa, Homo sapiens
MAVMAPRTLLLLLSGALALTQTWA

SEQ ID NO: 35 - protein, 24 aa, Homo sapiens
MAVMAPRTLVLLLSGALALTQTWA

SEQ ID NO: 36 - protein, 24 aa, Homo sapiens
MAVMAPRTLLLLLLGALALTQTWA

SEQ ID NO: 37 - protein, 24 aa, Homo sapiens
MAIMAPRTLVLLLSGALALTQTWA

SEQ ID NO: 38 - protein, 24 aa, Homo sapiens
MLVMAPRTVLLLLSAALALTETWA

SEQ ID NO: 39 - protein, 24 aa, Homo sapiens
MRVTAPRTVLLLLSGALALTETWA

SEQ ID NO: 40 - protein, 24 aa, Homo sapiens
MRVTAPRTVLLLLSAALALTETWA

SEQ ID NO: 41 - protein, 24 aa, Homo sapiens
MRVTAPRTLLLLLWGAVALTETWA

SEQ ID NO: 42 - protein, 24 aa, Homo sapiens
MRVTAPRTLLLLLWGALALTETWA

SEQ ID NO: 43 - protein, 24 aa, Homo sapiens
MRVTAPRTVLLLLWGAVALTETWA

SEQ ID NO: 44 - protein, 24 aa, Homo sapiens
MRVMAPRTLILLLSGALALTETWA

SEQ ID NO: 45 - protein, 24 aa, Homo sapiens
MRVMAPRALLLLLSGGLALTETWA

SEQ ID NO: 46 - protein, 24 aa, Homo sapiens
MRVMAPRTLLLLLSGALALTETWA

SEQ ID NO: 47 - protein, 24 aa, Homo sapiens
MRVMAPQALLLLLSGALALIETWA

SEQ ID NO: 48 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 49 - DNA, 22 bp, Artificial Sequence
  Forward primer
gggaccagacgggagattcctg

SEQ ID NO: 50 - DNA, 18 bp, Artificial Sequence
  Reverse primer
agctcgcatccgtgcatc

(19) United States
(12) Patent Application Publication
(10) Pub. No.: US 2024/0200031 A1
(43) Pub. Date: Jun. 20, 2024

(54) ENGINEERED HLA MOLECULES USEFUL FOR T CELL AND NK CELL ACTIVATION AND EXPANSION

(71) Applicant: Memorial Sloan Kettering Cancer Center, New York, NY (US)

(72) Inventors: Katharine C Hsu, New York, NY (US); Mohammed Kazim Panjwani, New York, NY (US)

(21) Appl. No.: 18/555,079

(22) PCT Filed: Apr. 12, 2022
(86) PCT No.: PCT/US2022/024435

§ 371 Date: Oct. 12, 2023

Related U.S. Application Data
(60) Provisional application No. 63/173,966, filed on Apr. 12, 2021.

(51) Int. Cl. C12N 5/0783; C07K 14/74; C12N 15/86

(52) U.S. Cl. CPC C12N 5/0636; C07K 14/70539; C12N 5/0646; C12N 15/86; C12N 2501/2302; C12N 2502/99; C12N 2510/00; C12N 2740/15043

(57) ABSTRACT

In some aspects, the present invention involves chimeric recombinant HLA proteins that are derived from HLA-E proteins and comprise a leader peptide from an HLA-A, HLA-B, HLA-C or HLA-G protein. In some aspects, the present invention also involves nucleic acid molecules encoding such recombinant HLA proteins, vectors comprising such nucleic acid molecules, host cells comprising such recombinant HLA proteins, and various related compositions. In some aspects, the present invention also involves methods of use of such recombinant HLA proteins, nucleic acid molecules, vectors, host cells and compositions.

### CROSS-REFERENCE TO RELATED APPLICATIONS

This application claims the benefit of priority of U.S. Provisional Patent Application No. 63/173,966 filed on Apr. 12, 2021, the content of which is hereby incorporated by reference in its entirety.

### STATEMENT REGARDING FEDERALLY SPONSORED RESEARCH

This invention was made with government support under grant number AI150999 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 12, 2022, is named MSKCC_053_WO1_ST25.txt and is 106,000 bytes in size.

### INCORPORATION BY REFERENCE

For the purpose of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND

The activating receptor NKG2C present on certain immune cells binds to an HLA-E:peptide complex. HLA-E is a non-classical class I HLA molecule that presents a nine amino acid-long peptide, typically derived from the signal peptide sequence of HLA-A, -B, -C, or -G that is expressed in the same cell. In general, the absence of HLA-A, -B, -C, and -G expression (or some other specific proteins), HLA-E does not come to the cell surface. To force expression of HLA-E on the surface of feeder cells used for NKG2C-based activation and/or expansion of T cells or natural killer (“NK”) cells, while avoiding expression of other class I HLA molecules, a surrogate peptide needs to be provided for presentation on the HLA-E molecule.

### SUMMARY OF THE INVENTION

The present invention involves recombinant HLA proteins. In particular, the present invention involves recombinant HLA proteins that are derived from HLA-E proteins but in which amino acids at the N-terminal end of the HLA-E protein are replaced with N-terminal amino acids derived from HLA-A, HLA-B, HLA-C or HLA-G proteins, such that the N-terminal leader peptide from HLA-A, HLA-B, HLA-C or HLA-G is present at the N-terminus of the HLA-E-derived recombinant HLA protein. Following proteolytic cleavage of the leader peptide and/or other intracellular processing, a 9 amino acid peptide (nonapeptide) from the leader peptide is available for loading into the HLA-E molecule, enabling expression of the HLA-E molecule on the cell surface and presentation of the loaded peptide to immune cells.

The present invention provides numerous of such novel recombinant HLA proteins, nucleic acid molecules encoding such recombinant HLA proteins, vectors comprising such nucleic acid molecules, host cells comprising such proteins, nucleic acid molecules, and/or vectors, and various compositions.

The present invention also provides various methods of use of such novel recombinant HLA proteins, nucleic acid molecules, vectors, host cells and compositions, including, but not limited to, methods in which host cells expressing the recombinant HLA proteins of the present invention are used to stimulate the expansion and or activation of immune cells such as T cells or NK cells.

FIG. 1 provides a schematic overview of some aspects of the present invention.

These and other aspects of the present invention are described further in the Detailed Description, Drawings, Brief Description of the Drawings, Examples and Claims sections of this patent disclosure. Furthermore, one of skill in the art will recognize that the various embodiments of the present invention described throughout this patent disclosure can be combined in various different ways, and that such combinations are within the scope of the present invention.

### BRIEF DESCRIPTION OF THE DRAWINGS

FIG. 1. Schematic illustration of the how the recombinant HLA proteins of the present invention are expressed, processed and presented on the surface of a host cell. The schematic illustration shows K562 host cells as an example. The illustration shows how, following transcription and translation of a nucleic acid molecule encoding a recombinant HLA protein as described herein, the leader peptide is cleaved off by a signal peptidase and the HLA-E molecule is expressed on the cell surface displaying a 9 amino acid peptide (nonapeptide) from the leader peptide. The illustration shows the leader peptide MVVMAPRTLFLLLSGALTLTETWA (amino acid residues 1-24 of SEQ ID NO. 1 and SEQ ID NO. 16) and the nonapeptide VMAPRTLFL (amino acid residues 3-11 of SEQ ID NO. 1 and SEQ ID NO. 16) as examples.

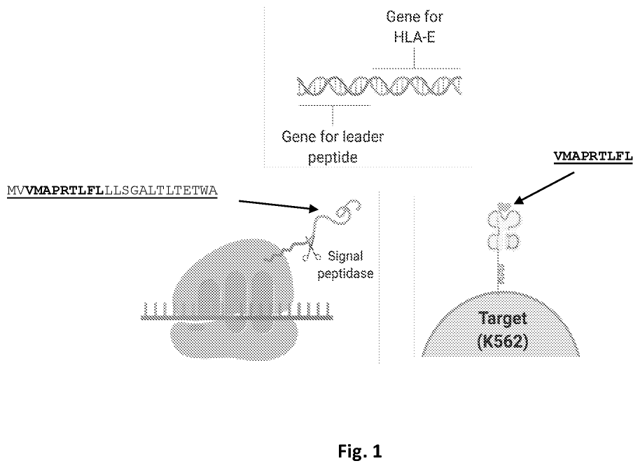

FIG. 2. The top panel provides an illustration of the amino acid sequence of HLA-E0103 (SEQ ID NO. 31) including its leader sequence (amino acid residues 1-21, underlined) and showing the location of the peptide cleavage site (indicated with an arrow). The lower panel provides an illustration of the amino acid sequence of the above HLA-E0103 molecule after removal of its 21 amino acid leader sequence. The amino acid sequence shown in the lower panel consists of amino acid residues 22-358 of SEQ ID No. 31.

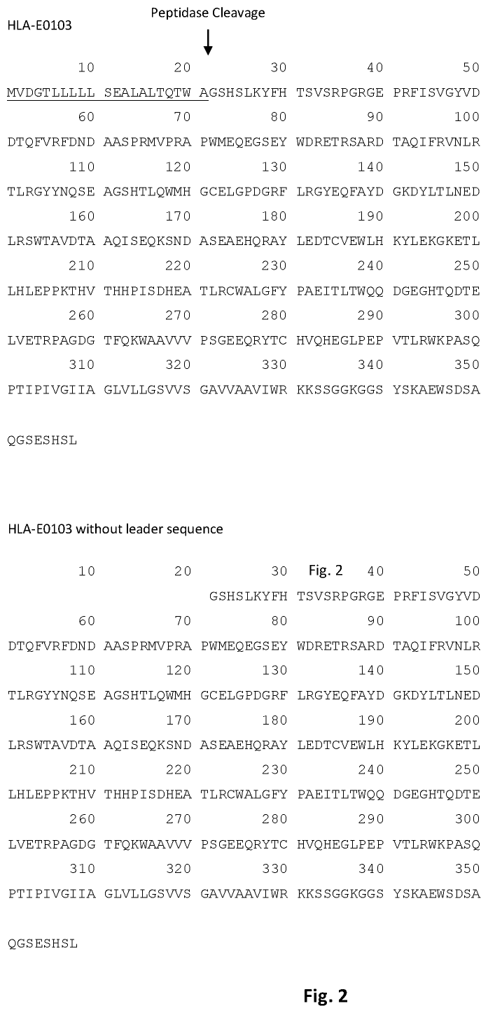

FIG. 3. The top panel provides an illustration of the amino acid sequence of HLA-E0101 (SEQ ID NO. 32) including its leader sequence (amino acid residues 1-21, underlined) and showing the location of the peptide cleavage site (indicated with an arrow). The lower panel provides an illustration of the amino acid sequence of the above HLA-E0101 molecule after removal of its 21 amino acid leader sequence. The amino acid sequence shown in the lower panel consists of amino acid residues 22-358 of SEQ ID No. 32.

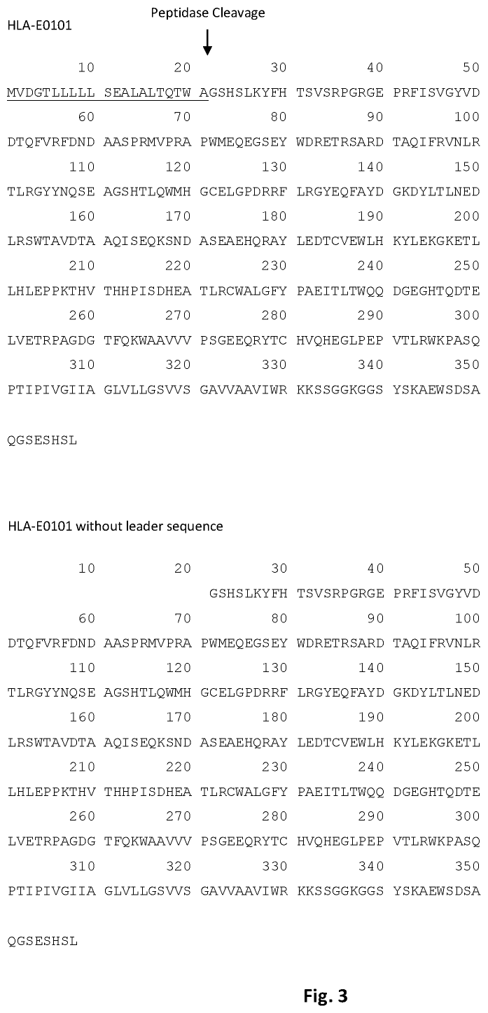

FIG. 4. Illustration of the amino acid sequence of HLA-G01 (SEQ ID NO. 48) including its leader sequence (amino acid residues 1-24, underlined) and showing the location of the peptide cleavage site (indicated with an arrow).

FIG. 5. The upper panel provides an illustration of the amino acid sequence of an example of a recombinant HLA protein according to the present invention in which the 21 amino acid leader peptide of HLA-E0103 (SEQ ID NO. 31) has been replaced with the 24 amino acid leader sequence SEQ ID NO. 33—which is the leader sequence of HLA-G01 (SEQ ID NO. 48). The lower panel provides an illustration of the amino acid sequence of another example of a recombinant HLA protein according to the present invention in which the 21 amino acid leader peptide of HLA-E0101 (SEQ ID NO. 32) has been replaced with the 24 amino acid leader sequence SEQ ID NO. 33—which is the leader sequence of HLA-G01 (SEQ ID NO. 48). As described elsewhere herein, the present invention provides other similar recombinant HLA proteins in which the leader peptide of an HLA-E molecule is replaced with the leader peptide of an HLA-A, HLA-B, HLA-C, or HLA-G molecule.

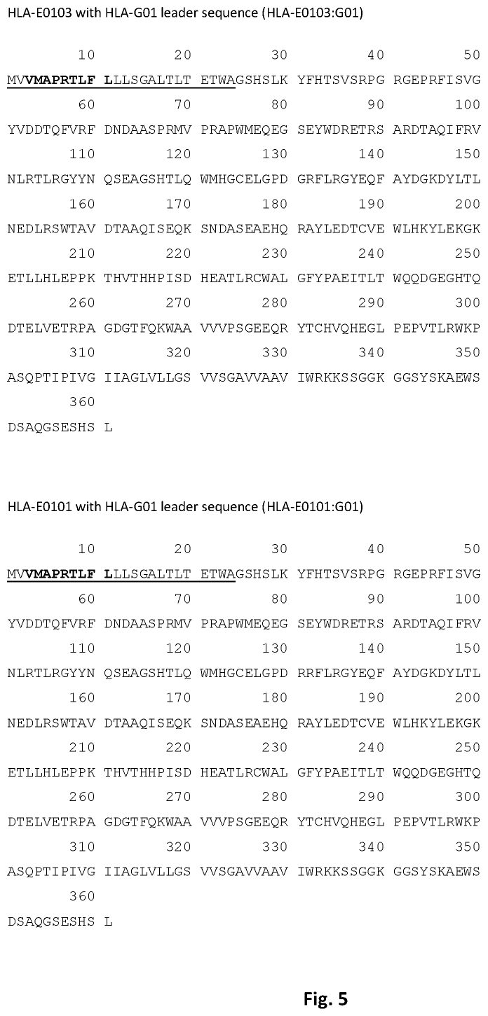

FIG. 6. Data from flow cytometry experiments performed using K562 cells, modified K562 cells expressing CD64, CD137L, CD86, truncated CD19, and mbIL-21 (“K562 mod”), 721.221 cells, and BAF3 cells. The middle row shows flow cytometry scatter plot data from non-transduced cells. The bottom row shows flow cytometry scatter plot data from cells transduced with a construct containing a nucleotide sequence encoding HLA-E0103:G01 (SEQ ID NO. 1). The top row provides overlay graphs showing the data from the non-transduced (dark gray) and transduced (light gray) cell populations.

FIG. 7. Graphs showing expression of HLA-E0103 chimeras comprising HLA-A01, HLA-A02, HLA-A31, HLA-A34, HLA-B15, or HLA-C15 full leader sequences in murine BAF3 cells expressing human beta-2 microglobulin. The bottom graph provides data from non-transduced cells.

FIG. 8. Upper panel—Graphs showing expression of HLA-E0103 chimeras having an HLA-A01, HLA-A02, HLA-A31, HLA-A34, HLA-B44, HLA-C04, HLA-C07, HLA-C15, HLA-C17, or HLA-G01 leader sequence in K562 cells. Lower panel—Graphs showing expression of HLA-E0103 chimeras having an HLA-B08 leader sequences in K562 cells.

FIG. 9. Graphs showing HLA-E expression in 721.221 cells transduced with an HLA-E0103 chimera having a leader sequence from HLA-A01, -A02, -A31, -A34, -B08, -C04, -C07, -C15, -C17, -G01, -B15, -B40, -B44, -B56, or B57 and non-transduced 721.221 cells.

FIG. 10. Graph showing NKG2A+ and NKG2C+ NK cell degranulation, as determined by CD107a+ mobilization, in response to exposure to K562 cells expressing two different HLA-E chimeras—HLA-E0103:G01 and HLA-E0103:A02. **** denotes statistical significance at the p<0.0001 level.

FIG. 11. Representative flow cytometry plots showing degranulation (CD107a) and intracellular IFN-g expression by NKG2C+ or NKG2C-CD8+ T cells after 6 hours stimulation with K562 host cells expressing HLA-E0103:G01. Graph on the right shows cumulative analysis of CD107a+ and IFN-γ+NKG2C+ or NKG2C-CD8+ T cells from 12 independent donors against the K562 host cells. Statistical significance was calculated using Wilcoxon matched-pairs signed-rank test.

FIG. 12. In vitro cytotoxicity of FACS-sorted NKG2C+ or NKG2C−CD8+ T cells against K562 host cells expressing HLA-E0103:G01 was assessed using a 6-hour bioluminescence assay. Results from three independent donors are shown. Statistical significance was calculated using a two-way ANOVA comparing the mean of each E:T ratio between NKG2C− and NKG2C+CD8+ T cells.

FIG. 13. NKG2C+CD8+ T cells can be generated in vitro from CD8+ T cells by BCL11B deletion and co-culture with K562 host cells expressing HLA-E0103: G01. The figure provides FACS plots showing the frequency of NKG2A+ versus NKG2C+ cells (upper panel) and CD56+ versus DAP12+ (lower panel) CD8+ T cells following culture of control and BCL11B KO CD8+ T cells with K562 host cells expressing HLA-E0103:G01 for 2 weeks or 4 weeks.

### DETAILED DESCRIPTION OF THE INVENTION

In order that the present invention can be more readily understood, certain terms are defined below. Additional definitions are set forth throughout the disclosure. Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Wherever embodiments are described with the language “comprising,” otherwise analogous embodiments described in terms of “consisting of” and/or “consisting essentially of” are included.

Amino acids are referred to herein by their commonly known one-letter symbols recommended by the IUPAC-IUB Biochemical Nomenclature Commission.

The abbreviation “HLA” refers to human leukocyte antigen.

The term “vector” means a construct for delivery of a nucleic acid molecule to a cell. Examples of vectors include, but are not limited to, viruses, viral-derived vectors, naked DNA or RNA vectors, plasmid vectors, cosmid vectors, phage vectors, and the like. In some embodiments a vector may be an “expression vector” that is capable of delivering a nucleic acid molecule to a cell and that also contains elements required for expression of the nucleic acid molecule in the cell.

The term “nucleic acid molecule” as used herein includes both DNA molecules and RNA molecules. Similarly, the term “nucleic acid sequence” as used herein includes both DNA sequences and RNA sequences.

The terms “signal peptide,” “signal sequence,” “leader peptide,” and “leader sequence” are used interchangeably herein.

In each of the embodiments described herein, the recombinant HLA proteins and/or nucleic acid molecules and/or cells (or populations of cells) can optionally be in “isolated” form. An “isolated” protein or nucleic acid molecule or cell (or population of cells) is not within a living subject (or, in the case of proteins and/or nucleic acid molecules, is not within a cell) and is typically in a form not found in nature. In some embodiments an isolated protein or nucleic acid molecule or cell (or populations of cells) may be purified to a degree that it is not in a form in which it is found in nature. In some embodiments, an isolated protein or nucleic acid molecule or cell (or populations of cells) is substantially pure. In some embodiments, an isolated protein or nucleic acid molecule or cell (or populations of cells) has a purity of greater than 75%, or greater than 80%, or greater than 90%, or greater than 95% of the stated protein or nucleic acid molecule or cell (or populations of cells).

The terms “identical” or percent “identity” in the context of two or more amino acid sequences, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

Other terms are defined elsewhere in this patent disclosure, or else are used in accordance with their usual meaning in the art.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Ausubel et al. eds. (2015) Current Protocols in Molecular Biology (John Wiley and Sons); Greenfield, ed. (2013) Antibodies: A Laboratory Manual (2nd ed., Cold Spring Harbor Press); Green and Sambrook, eds. (2012), Molecular Cloning: A Laboratory Manual (4th ed., Cold Spring Harbor Laboratory Press); Krebs et al., eds. (2012) Lewin's Genes XI (11th ed., Jones & Bartlett Learning); Freshney (2010) Culture Of Animal Cells (6th ed., Wiley); Weir and Blackwell, eds., (1996) Handbook Of Experimental Immunology, Volumes I-IV (5th ed., Wiley-Blackwell); Borrebaeck, ed. (1995) Antibody Engineering (2nd ed., Oxford Univ. Press); Glover and Hames, eds., (1995) DNA Cloning: A Practical Approach, Volumes I and II (2nd ed., IRL Press); Rees et al., eds. (1993) Protein Engineering: A Practical Approach (1st ed., IRL Press); Mayer and Walker, eds. (1987) Immunochemical Methods In Cell And Molecular Biology (Academic Press, London); Nisonoff (1984) Introduction to Molecular Immunology (2nd ed., Sinauer Associates, Inc.); and Steward (1984) Antibodies: Their Structure and Function (1st ed., Springer Netherlands).

### Recombinant HLA Proteins

The present invention provides recombinant HLA proteins. In particular, the present invention provides recombinant HLA proteins that are modified HLA-E proteins in which amino acids of the leader peptide at the N-terminal end of the HLA-E protein are replaced with the amino acids of the N-terminal leader peptide from HLA-A, HLA-B, HLA-C or HLA-G proteins. In such proteins, the leader peptide from HLA-A, HLA-B, HLA-C or HLA-G is present at the N-terminus of the recombinant HLA protein and, after proteolytic cleavage of the signal peptide and/or other post-translational processing, a 9 amino acid peptide within the signal peptide is available for loading into the HLA-E molecule.

Accordingly, in some embodiments the present invention provides recombinant HLA proteins comprising, from N-terminal to C-terminal, a leader peptide from an HLA-A, HLA-B, HLA-C, or HLA-G molecule and an HLA-E protein, wherein the recombinant HLA protein does not comprise an HLA-E leader peptide. In some embodiments there are no intervening amino acids between the leader peptide and the HLA-E protein. In some embodiments there is no linker moiety between the leader peptide and the HLA-E protein.

In some embodiments the leader peptide from HLA-A, HLA-B, HLA-C, or HLA-G comprises amino acids 3-11 of any one of SEQ ID NOs. 33-47 (i.e., the 9 amino acid nonapeptide from SEQ ID NOs. 33-47 that can be loaded onto an HLA-E protein for presentation to an immune cell).

In some embodiments the leader peptide from HLA-A, HLA-B, HLA-C, or HLA-G comprises the amino acid sequence of any one of SEQ ID NOs. 33-47.

In some embodiments the HLA-E protein is derived from the amino acid sequence of SEQ ID NO. 31 or SEQ ID NO. 32.

In some embodiments the HLA-E protein comprises amino acids 22-358 of SEQ ID NO. 31 or SEQ ID NO. 32.

In some embodiments the recombinant HLA protein comprises any one of SEQ ID NOs. 1 to 30. For example, in one embodiment the present invention provides a recombinant HLA protein comprising SEQ ID NO. 1. In another embodiment the present invention provides a recombinant HLA protein comprising SEQ ID NO. 2. In another embodiment the present invention provides a recombinant HLA protein comprising SEQ ID NO. 3. In another embodiment the present invention provides a recombinant HLA protein comprising SEQ ID NO. 4. In another embodiment the present invention provides a recombinant HLA protein comprising SEQ ID NO. 5. In another embodiment the present invention provides a recombinant HLA protein comprising SEQ ID NO. 6. In another embodiment the present invention provides a recombinant HLA protein comprising SEQ ID NO. 7. In another embodiment the present invention provides a recombinant HLA protein comprising SEQ ID NO. 8. In another embodiment the present invention provides a recombinant HLA protein comprising SEQ ID NO. 9. In another embodiment the present invention provides a recombinant HLA protein comprising SEQ ID NO. 10. In another embodiment the present invention provides a recombinant HLA protein comprising SEQ ID NO. 11. In another embodiment the present invention provides a recombinant HLA protein comprising SEQ ID NO. 12. In another embodiment the present invention provides a recombinant HLA protein comprising SEQ ID NO. 13. In another embodiment the present invention provides a recombinant HLA protein comprising SEQ ID NO. 14. In another embodiment the present invention provides a recombinant HLA protein comprising SEQ ID NO. 15. In another embodiment the present invention provides a recombinant HLA protein comprising SEQ ID NO. 16. In another embodiment the present invention provides a recombinant HLA protein comprising SEQ ID NO. 17. In another embodiment the present invention provides a recombinant HLA protein comprising SEQ ID NO. 18 In another embodiment the present invention provides a recombinant HLA protein comprising SEQ ID NO. 19. In another embodiment the present invention provides a recombinant HLA protein comprising SEQ ID NO. 20. In another embodiment the present invention provides a recombinant HLA protein comprising SEQ ID NO. 21. In another embodiment the present invention provides a recombinant HLA protein comprising SEQ ID NO. 22. In another embodiment the present invention provides a recombinant HLA protein comprising SEQ ID NO. 23. In another embodiment the present invention provides a recombinant HLA protein comprising SEQ ID NO. 24. In another embodiment the present invention provides a recombinant HLA protein comprising SEQ ID NO. 25. In another embodiment the present invention provides a recombinant HLA protein comprising SEQ ID NO. 26. In another embodiment the present invention provides a recombinant HLA protein comprising SEQ ID NO. 27. In another embodiment the present invention provides a recombinant HLA protein comprising SEQ ID NO. 28. In another embodiment the present invention provides a recombinant HLA protein comprising SEQ ID NO. 29. In another embodiment the present invention provides a recombinant HLA protein comprising SEQ ID NO. 30.

The recombinant HLA proteins described above are processed intracellularly leading to cell-surface HLA expression and peptide presentation. In some embodiments the present invention provides recombinant HLA proteins produced intracellularly from any one of the proteins described above, for example by proteolytic cleavage of its leader peptide.

The amino acid sequences of 30 examples of recombinant HLA proteins according to the present invention listed above, as identified by their SEQ ID NOs, are provided in Table A, below. In Table A, the amino acids of the leader sequence, which is derived from another HLA molecule (i.e., not from HLA-E), are bolded (i.e., the first 24 amino acids), and the portion of the leader peptide that constitutes the 9 amino acid peptide that is loaded into and displayed on the HLA-E-derived molecule is underlined (i.e., amino acids 3-11).

TABLE A

| | SEQ | |
|---|---|---|
| NAME | ID NO. | AMINO ACID SEQUENCE |
| HLA-E0103:G01 | 1 | MVVMAPRTLFLLLSGALTLTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:A01 | 2 | MAVMAPRTLLLLLSGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:A02 | 3 | MAVMAPRTLVLLLSGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:A31 | 4 | MAVMAPRTLLLLLLGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:A34 | 5 | MAIMAPRTLVLLLSGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:B08 | 6 | MLVMAPRTVLLLLSAALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:B15 | 7 | MRVTAPRTVLLLLSGALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E:B40 | 8 | MRVTAPRTVLLLLSAALALTETWAGSHSLKYFHTSVSRPGRGEPRFISVGYVDD |
| | | TQFVRFDNDAASPRMVPRAPWMEQEGSEYWDRETRSARDTAQIFRVNLRTLRG |
| | | YYNQSEAGSHTLQWMHGCELGPDGRFLRGYEQFAYDGKDYLTLNEDLRSWTAV |
| | | DTAAQISEQKSNDASEAEHQRAYLEDTCVEWLHKYLEKGKETLLHLEPPKTHVT |
| | | HHPISDHEATLRCWALGFYPAEITLTWQQDGEGHTQDTELVETRPAGDGTFQKW |
| | | AAVVVPSGEEQRYTCHVQHEGLPEPVTLRWKPASQPTIPIVGIIAGLVLLGSVVSG |
| | | AVVAAVIWRKKSSGGKGGSYSKAEWSDSAQGSESHSL |
| HLA-E0103:B44 | 9 | MRVTAPRTLLLLLWGAVALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E:B56 | 10 | MRVTAPRTLLLLLWGALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:B57 | 11 | MRVTAPRTVLLLLWGAVALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:C04 | 12 | MRVMAPRTLILLLSGALALTETWAGSHSLKYFHTSVSRPGRGEPRFISVGYVDD |
| | | TQFVRFDNDAASPRMVPRAPWMEQEGSEYWDRETRSARDTAQIFRVNLRTLRG |
| | | YYNQSEAGSHTLQWMHGCELGPDGRFLRGYEQFAYDGKDYLTLNEDLRSWTAV |
| | | DTAAQISEQKSNDASEAEHQRAYLEDTCVEWLHKYLEKGKETLLHLEPPKTHVT |
| | | HHPISDHEATLRCWALGFYPAEITLTWQQDGEGHTQDTELVETRPAGDGTFQKW |
| | | AAVVVPSGEEQRYTCHVQHEGLPEPVTLRWKPASQPTIPIVGIIAGLVLLGSVVSG |
| | | AVVAAVIWRKKSSGGKGGSYSKAEWSDSAQGSESHSL |
| HLA-E0103:C07 | 13 | MRVMAPRALLLLLSGGLALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:C15 | 14 | MRVMAPRTLLLLLSGALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0103:C17 | 15 | MRVMAPQALLLLLSGALALIETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDGRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:G01 | 16 | MVVMAPRTLFLLLSGALTLTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:A01 | 17 | MAVMAPRTLLLLLSGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:A02 | 18 | MAVMAPRTLVLLLSGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:A31 | 19 | MAVMAPRTLLLLLLGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:A34 | 20 | MAIMAPRTLVLLLSGALALTQTWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:B08 | 21 | MLVMAPRTVLLLLSAALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:B15 | 22 | MRVTAPRTVLLLLSGALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:B40 | 23 | MRVTAPRTVLLLLSAALALTETWAGSHSLKYFHTSVSRPGRGEPRFISVGYVDD |
| | | TQFVRFDNDAASPRMVPRAPWMEQEGSEYWDRETRSARDTAQIFRVNLRTLRG |
| | | YYNQSEAGSHTLQWMHGCELGPDRRFLRGYEQFAYDGKDYLTLNEDLRSWTAV |
| | | DTAAQISEQKSNDASEAEHQRAYLEDTCVEWLHKYLEKGKETLLHLEPPKTHVT |
| | | HHPISDHEATLRCWALGFYPAEITLTWQQDGEGHTQDTELVETRPAGDGTFQKW |
| | | AAVVVPSGEEQRYTCHVQHEGLPEPVTLRWKPASQPTIPIVGIIAGLVLLGSVVSG |
| | | AVVAAVIWRKKSSGGKGGSYSKAEWSDSAQGSESHSL |
| HLA-E0101:B44 | 24 | MRVTAPRTLLLLLWGAVALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:B56 | 25 | MRVTAPRTLLLLLWGALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:B57 | 26 | MRVTAPRTVLLLLWGAVALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:C04 | 27 | MRVMAPRTLILLLSGALALTETWAGSHSLKYFHTSVSRPGRGEPRFISVGYVDD |
| | | TQFVRFDNDAASPRMVPRAPWMEQEGSEYWDRETRSARDTAQIFRVNLRTLRG |
| | | YYNQSEAGSHTLQWMHGCELGPDRRFLRGYEQFAYDGKDYLTLNEDLRSWTAV |
| | | DTAAQISEQKSNDASEAEHQRAYLEDTCVEWLHKYLEKGKETLLHLEPPKTHVT |
| | | HHPISDHEATLRCWALGFYPAEITLTWQQDGEGHTQDTELVETRPAGDGTFQKW |
| | | AAVVVPSGEEQRYTCHVQHEGLPEPVTLRWKPASQPTIPIVGIIAGLVLLGSVVSG |
| | | AVVAAVIWRKKSSGGKGGSYSKAEWSDSAQGSESHSL |
| HLA-E0101:C07 | 28 | MRVMAPRALLLLLSGGLALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:C15 | 29 | MRVMAPRTLLLLLSGALALTETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL |
| HLA-E0101:C17 | 30 | MRVMAPQALLLLLSGALALIETWAGSHSLKYFHTSVSRPGRGEPRFISVGYVD |
| | | DTQFVRFDNDAASPRMVPRAPWMEQEGSEYWDRETRSARDTAQIFRVNLRTLR |
| | | GYYNQSEAGSHTLQWMHGCELGPDRRFLRGYEQFAYDGKDYLTLNEDLRSWTA |
| | | VDTAAQISEQKSNDASEAEHQRAYLEDTCVEWLHKYLEKGKETLLHLEPPKTHV |
| | | THHPISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGSVVS |
| | | GAVVAAVIWRKKSSGGKGGSYSKAEWSDSAQGSESHSL/ |

The amino acid sequences of the HLA-E proteins from which the recombinant HLA proteins listed in Table A are derived are provided in Table B, below. In Table B, the amino acids of the leader sequence are bolded (i.e., the first 21 amino acids). These are the amino acids that are substituted with leader peptides from a different HLA molecule (HLA-A, HLA-B, HLA-C, or HLA-G) in the recombinant HLA proteins shown in Table A.

TABLE B

| | SEQ | |
|---|---|---|
| NAME | ID NO. | AMINO ACID SEQUENCE |
| HLA-E0103 | 31 | MVDGTLLLLLSEALALTQTWAGSHSLKYFHTSVSRPGRGEPRFISVGYVDDTQF |
| | | VRFDNDAASPRMVPRAPWMEQEGSEYWDRETRSARDTAQIFRVNLRTLRGYYN |
| | | QSEAGSHTLQWMHGCELGPDGRFLRGYEQFAYDGKDYLTLNEDLRSWTAVDTA |
| | | AQISEQKSNDASEAEHQRAYLEDTCVEWLHKYLEKGKETLLHLEPPKTHVTHHPI |
| | | SDHEATLRCWALGFYPAEITLTWQQDGEGHTQDTELVETRPAGDGTFQKWAAV |
| | | VVPSGEEQRYTCHVQHEGLPEPVTLRWKPASQPTIPIVGIIAGLVLLGSVVSGAVV |
| | | AAVIWRKKSSGGKGGSYSKAEWSDSAQGSESHSL |
| HLA-E0101 | 32 | MVDGTLLLLLSEALALTQTWAGSHSLKYFHTSVSRPGRGEPRFISVGYVDDTQF |
| | | VRFDNDAASPRMVPRAPWMEQEGSEYWDRETRSARDTAQIFRVNLRTLRGYYN |
| | | QSEAGSHTLQWMHGCELGPDRRFLRGYEQFAYDGKDYLTLNEDLRSWTAVDTA |
| | | AQISEQKSNDASEAEHQRAYLEDTCVEWLHKYLEKGKETLLHLEPPKTHVTHHPI |
| | | SDHEATLRCWALGFYPAEITLTWQQDGEGHTQDTELVETRPAGDGTFQKWAAV |
| | | VVPSGEEQRYTCHVQHEGLPEPVTLRWKPASQPTIPIVGIIAGLVLLGSVVSGAVV |
| | | AAVIWRKKSSGGKGGSYSKAEWSDSAQGSESHSL |

Amino acid sequences of leader peptides from various HLA-A, HLA-B, HLA-C, and HLA-G types, which can be used to replace the leader peptides of HLA-E molecules, are provided in Table C, below. Amino acid residues 3-11 of each of these leader peptides (underlined) constitutes a 9 amino acid peptide (nonapeptide) that can be loaded into an HLA-A molecule and presented on the cell surface of a host cell.

TABLE C

| HLA | SEQ | AMINO ACID |
|---|---|---|
| Type | ID NO. | SEQUENCE OF LEADER PEPTIDE |
| HLA-G01 | 33 | MVVMAPRTLFLLLSGALTLTETWA |
| HLA-A01 | 34 | MAVMAPRTLLLLLSGALALTQTWA |
| HLA-A02 | 35 | MAVMAPRTLVLLLSGALALTQTWA |
| HLA-A31 | 36 | MAVMAPRTLLLLLLGALALTQTWA |
| HLA-A34 | 37 | MAIMAPRTLVLLLSGALALTQTWA |
| HLA-B08 | 38 | MLVMAPRTVLLLLSAALALTETWA |
| HLA-B15 | 39 | MRVTAPRTVLLLLSGALALTETWA |
| HLA-B40 | 40 | MRVTAPRTVLLLLSAALALTETWA |
| HLA-B44 | 41 | MRVTAPRTLLLLLWGAVALTETWA |
| HLA-B56 | 42 | MRVTAPRTLLLLLWGALALTETWA |
| HLA-B57 | 43 | MRVTAPRTVLLLLWGAVALTETWA |
| HLA-C04 | 44 | MRVMAPRTLILLLSGALALTETWA |
| HLA-C07 | 45 | MRVMAPRALLLLLSGGLALTETWA |
| HLA-C15 | 46 | MRVMAPRTLLLLLSGALALTETWA |
| HLA-C17 | 47 | MRVMAPQALLLLLSGALALIETWA |

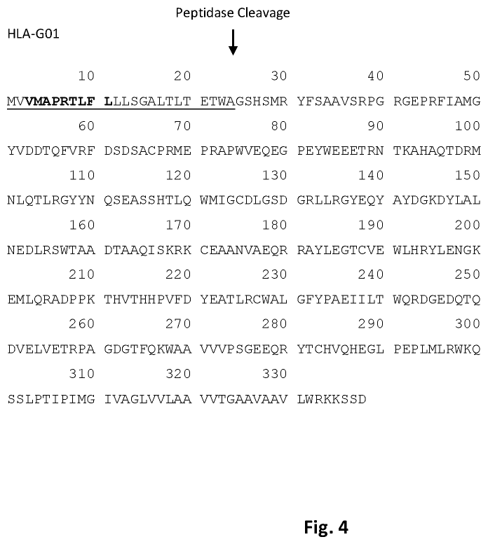

The full amino acid sequence of HLA-G01 (SEQ ID NO. 48) is illustrated in FIG. 4

In some embodiments the present invention provides variants of the recombinant HLA proteins described above or elsewhere herein. For example, in some embodiments the present 5 invention provides variants of the recombinant HLA proteins having the amino acid sequences of any of SEQ ID Nos 1-30. In some embodiments the present invention provides variants of the recombinant HLA proteins described above or elsewhere herein that comprise 1 or 2 or 3 or 4 or 5 or more amino acid point mutations (which can be insertions, deletions, or substitutions) as compared to the recombinant HLA proteins described above or elsewhere 10 herein, or have an amino acid sequence that is at least 90%, or at least 95%, or at least 96%, or at least 97%, or at least 98%, or at least 99% identical to that of any of the recombinant HLA proteins described above or elsewhere herein, provided that such variant recombinant HLA proteins can be expressed on the surface of a host cell and can be loaded with and present a nonapeptide derived from their leader peptide.

### Nucleic Acid Molecules

The present invention also provides nucleic acid molecules that encode any of the recombinant HLA proteins described herein. In some embodiments such nucleic acid molecules are DNA molecules. In some embodiments such nucleic acid molecules are RNA molecules.

For example, in one embodiment the present invention provides a nucleic acid molecule that encodes a recombinant HLA protein comprising any one of SEQ ID NOs. 1 to 30. In one embodiment the present invention provides a nucleic acid molecule that encodes a recombinant HLA protein comprising SEQ ID NO. 1. In another embodiment the present invention provides a nucleic acid molecule that encodes a recombinant HLA protein comprising SEQ ID NO. 2. In another embodiment the present invention provides a nucleic acid molecule that encodes a recombinant HLA protein comprising SEQ ID NO. 3. In another embodiment the present invention provides a nucleic acid molecule that encodes a recombinant HLA protein comprising SEQ ID NO. 4. In another embodiment the present invention provides a nucleic acid molecule that encodes a recombinant HLA protein comprising SEQ ID NO. 5. In another embodiment the present invention provides a nucleic acid molecule that encodes a recombinant HLA protein comprising SEQ ID NO. 6. In another embodiment the present invention provides a nucleic acid molecule that encodes a recombinant HLA protein comprising SEQ ID NO. 7. In another embodiment the present invention provides a nucleic acid molecule that encodes a recombinant HLA protein comprising SEQ ID NO. 8. In another embodiment the present invention provides a nucleic acid molecule that encodes a recombinant HLA protein comprising SEQ ID NO. 9. In another embodiment the present invention provides a nucleic acid molecule that encodes a recombinant HLA protein comprising SEQ ID NO. 10. In another embodiment the present invention provides a nucleic acid molecule that encodes a recombinant HLA protein comprising SEQ ID NO. 11. In another embodiment the present invention provides a nucleic acid molecule that encodes a recombinant HLA protein comprising SEQ ID NO.12. In another embodiment the present invention provides a nucleic acid molecule that encodes a recombinant HLA protein comprising SEQ ID NO. 13. In another embodiment the present invention provides a nucleic acid molecule that encodes a recombinant HLA protein comprising SEQ ID NO. 14. In another embodiment the present invention provides a nucleic acid molecule that encodes a recombinant HLA protein comprising SEQ ID NO. 15. In another embodiment the present invention provides a nucleic acid molecule that encodes a recombinant HLA protein comprising SEQ ID NO. 16. In another embodiment the present invention provides a nucleic acid molecule that encodes a recombinant HLA protein comprising SEQ ID NO. 17. In another embodiment the present invention provides a nucleic acid molecule that encodes a recombinant HLA protein comprising SEQ ID NO. 18. In another embodiment the present invention provides a nucleic acid molecule that encodes a recombinant HLA protein comprising SEQ ID NO. 19. In another embodiment the present invention provides a nucleic acid molecule that encodes a recombinant HLA protein comprising SEQ ID NO. 20. In another embodiment the present invention provides a nucleic acid molecule that encodes a recombinant HLA protein comprising SEQ ID NO. 21. In another embodiment the present invention provides a nucleic acid molecule that encodes a recombinant HLA protein comprising SEQ ID NO. 22. In another embodiment the present invention provides a nucleic acid molecule that encodes a recombinant HLA protein comprising SEQ ID NO. 23. In another embodiment the present invention provides a nucleic acid molecule that encodes a recombinant HLA protein comprising SEQ ID NO. 24. In another embodiment the present invention provides a nucleic acid molecule that encodes a recombinant HLA protein comprising SEQ ID NO. 25. In another embodiment the present invention provides a nucleic acid molecule that encodes a recombinant HLA protein comprising SEQ ID NO. 26. In another embodiment the present invention provides a nucleic acid molecule that encodes a recombinant HLA protein comprising SEQ ID NO. 27. In another embodiment the present invention provides a nucleic acid molecule that encodes a recombinant HLA protein comprising SEQ ID NO. 28. In another embodiment the present invention provides a nucleic acid molecule that encodes a recombinant HLA protein comprising SEQ ID NO. 29. In another embodiment the present invention provides a nucleic acid molecule that encodes a recombinant HLA protein comprising SEQ ID NO. 30.

In some embodiments the nucleic acid molecules are codon optimized for human expression.

In some embodiments the nucleic acid molecules are operatively linked to a promoter. In some such embodiments the promoter is the EF1α promoter.

### Vectors

The present invention also provides vectors that comprise any of the nucleic molecules described herein. In some of such embodiments the vectors are cloning vectors. In some of such embodiments the vectors are expression vectors. In some of such embodiments the vectors are viral vectors. In some of such embodiments the vectors are lentiviral vectors.

### Host Cells

The present invention also provides cells that comprise a nucleic acid molecule that encodes a recombinant HLA protein as described herein, and/or that express a recombinant HLA protein encoded by such a nucleic acid molecule. Such cells are referred to herein as “host cells.” The host cells can be any cell type capable of expressing the recombinant HLA molecules of the present invention on their cell surface.

In some embodiments the host cells are mammalian cells. In some embodiments the host cells are human cells. In some embodiments the host cells are in vitro. In some embodiments the host cells are in vivo.

In some embodiments the host cells are K562 cells. See Lozzi et al., Blood, 1975 March; 45(3), pp. 321-34 for a description of K562 cells, which are well known and widely used in the art. In some embodiments the host cells are K562 cells modified to express 4-1BBL. In some embodiments the host cells are K526 cells modified to express membrane-bound IL-21 (mbIL-21). In some embodiments the host cells are K526 cells modified to express both 41BBL and mbIL-21. In some embodiments the host cells are the K562-derived cell line K562 c9.mbIL-21 (which is modified to modified to express CD64, CD86, CD137L, truncated CD19, eGFP, and membrane-bound IL-21, as described in the Examples section of this disclosure). See Singh et al. Cancer Res. 2011 May 15; 71(10), pp. 3516-27 for a description of modified K562 cells.

In some embodiments the host cells are the K562-derived cell line K562 c9.mbIL-21.E:G01 (described in the Examples section of this disclosure).

In some embodiments the host cells are 721.221 cells. See Shimizu et al., (1988). Proceedings of the National Academy of Sciences of the United States of America, 85(1), pp. 227-231 for a description of 721.221 cells.

In some embodiments the host cells are BAF3 cells. See Palacios et al., Nature. 1984 May 10-16; 309(5964), pp. 126-31 for a description of BAF3 cells.

In embodiments where the host cells are contacted in vitro (e.g., co-cultured) with immune cells, such as T cells or NK cells, the host cells may be referred to as “feeder” cells.

In some embodiments the host cells are isolated cells.

### Compositions

In some embodiments the present invention provides compositions that comprise a recombinant HLA protein as described herein, or a nucleic acid molecule encoding a recombinant HLA protein as described herein, or a host cell (or population of host cells) as described herein, and at least one additional component.

Examples of such additional components include diluents, saline solutions, cell culture media, buffers, carriers, stabilizers, dispersing agents, suspending agents, thickening agents, excipients, preservatives, and the like. In some embodiments the compositions are sterile.

In some embodiments the compositions are pharmaceutical compositions. The term “pharmaceutical composition” as used herein refers to a composition that is in such form as to permit the biological activity of the recombinant HLA protein, or nucleic acid molecule, or host cell (or population of host cells) therein, and which contains no components that are unacceptably toxic to cells or to living subjects.

### Methods of Production

The recombinant HLA proteins of the present invention can be produced by any of the methods known in the art for production of recombinant proteins. For example, in some embodiments the recombinant HLA proteins of the present invention are produced by expression in a host cell from a recombinant nucleic acid molecule that encodes the recombinant HLA protein. In some embodiments the recombinant nucleic acid molecule is a DNA molecule. In some embodiments the recombinant nucleic acid molecule is an RNA molecule.

Similarly, nucleic acid molecules encoding the recombinant HLA proteins of the present invention can be produced by any of the methods known in the art for production of nucleic acid molecules, such as standard methods of molecular biology and/or recombinant DNA technology.

### Methods of Use

The present invention also provides methods of use of the recombinant HLA proteins, nucleic acid molecules and host cells described herein.

In some embodiments the present invention provides methods for stimulating the expansion of immune cells. In some embodiments the present invention provides methods for modulating the activity of immune cells. In some embodiments the present invention provides methods for activating immune cells. In some embodiments the present invention provides methods for generating de novo populations of immune cells. In some embodiments the present invention provides methods of generating or preparing immune cells for use in adoptive cell therapy. In some embodiments the present invention provides methods of generating or preparing immune cells for administration to a subject, such as a human subject.

In some embodiments such methods involve contacting the immune cells with a recombinant HLA protein as described herein. In some embodiments such methods involve contacting the immune cells with a host cell expressing a recombinant HLA protein as described herein. In some embodiments such methods involve delivering a nucleic acid molecule encoding a recombinant HLA protein as described herein to a host cell. In some embodiments such methods involve delivering a vector comprising a nucleic acid molecule encoding a recombinant HLA protein as described herein to a host cell. In some embodiments such methods involve delivering a composition comprising a recombinant HLA protein, nucleic acid molecule or vector as described herein to a host cell.

In some embodiments, the immune cells are T cells. In some embodiments the T cells are CD8+ T cells. In some embodiments the T cells are NKG2C+CD8+ T cells. In some embodiments the T cells are NKG2C-CD8+ T cells. In some embodiments the T cells are CD8+ T cells in which the BCL11B gene has been knocked out (“BCL11B KO” cells).

In some embodiments the immune cells are NK cells.

For example, and as demonstrated experimentally in the Examples section of this patent disclosure, in some embodiments the present invention provides methods of activating NK cells by contacting the NK cells in vitro with a host cell expressing a recombinant HLA protein as described herein.

Similarly, and as also demonstrated experimentally in the Examples section of this patent disclosure, in some embodiments the present invention provides methods of activating T cells by contacting the T cells in vitro with a host cell expressing a recombinant HLA protein as described herein.

Similarly, in some embodiments the present invention provides methods of inducing de novo T cell populations by contacting T cells in vitro with a host cell expressing a recombinant HLA protein as described herein. For example, and as described in the Examples section of this patent disclosure, the present invention provides methods of inducing de novo NKG2C+ CD8+ T cell populations by contacting CD8+ T cells in which the BCL11B gene has been knocked out (“BCL11B KO” cells) in vitro with a host cell expressing a recombinant HLA protein as described herein.

In some embodiments the contacting is in vitro. In some embodiments the contacting is in vivo. Embodiments that involve contacting immune cells with a host cell expressing a recombinant HLA protein as described herein in vitro may be referred to herein as “feeder cell” methods or “co-culture methods” and the host cells used in such methods may be referred to as “feeder cells.”

The ability to stimulate the expansion of immune cells and/or modulate the activity of immune cells and/or induce a de novo immune cell population by contacting immune cells in vitro with a host cell expressing a recombinant HLA protein, as described herein, has several important clinical applications, including, but not limited to, in providing expanded and/or activated immune cells or de novo immune cell populations for use in adoptive cell therapy methods and/or other therapeutic methods in which immune cells (e.g., T cells or NK cells) are administered to subjects. Accordingly, in some embodiments, the methods described herein are used in conjunction with or as part of a method of generating or preparing immune cells for use in adoptive cell therapy. In some embodiments, the methods described herein are used in conjunction with or as part of a method of generating or preparing immune cells for administration to a subject, such as a human subject.

These and other embodiments of the invention are further described in the “Examples” section of this patent disclosure. All sections of this patent disclosure are intended to be read in conjunction with, and in the context of, all other sections of the present patent disclosure. Furthermore, one of skill in the art will recognize that the various embodiments of the present invention described herein can be combined in various ways, and that such combinations are within the scope of the present invention.

The invention is further described by, and understood with reference to, the following non-limiting Example(s).

### EXAMPLES

The activating receptor NKG2C binds to HLA-E:peptide complexes. HLA-E is a non-classical class I HLA molecule that presents a nine amino acid-long peptide, typically derived from the signal peptide sequence of HLA-A, -B, -C, or -G that is expressed in the same cell. In general, in the absence of HLA-A, -B, -C, and -G expression (or some other specific proteins), HLA-E does not come to the cell surface. To force expression of HLA-E on the surface of cells (e.g, feeder cells used for activation of immune cells via NKG2C) while avoiding expression of other class I HLA molecules, a surrogate peptide (e.g., a nine amino acid peptide from the signal peptide sequence of HLA-A, -B, -C, or -G) needs to be provided alongside HLA-E.

The present invention provides recombinant HLA proteins that are chimeric molecules derived from HLA-E molecules but in which the HLA-E signal peptide (also referred to as leader peptide) sequence has been replaced with a signal peptide from an HLA-A, -B, -C, or G molecule.

### Example 1

### Materials & Methods

K562 derivative cell lines are frequently used for expanding T cells and NK cells. In some experiments the chimeric proteins were expressed in K562s modified to express CD64, CD86, CD137L, truncated CD19, eGFP, and membrane-bound IL-21, referred to as “K562 c9.mbIL-21” cells.

Nucleotide sequences encoding the recombinant HLA proteins described herein were codon-optimized for human translation and synthesized in a pUC57 vector. Site-directed mutagenesis was used to convert HLA-E0101 sequences to HLA-E0103 sequences (i.e., to introduce an R128G mutation) using the Q5 Site-Directed Mutagenesis Kit [New England Biolabs (NEB)] with the forward primer 5′-GGGACCAGACGGGAGATTCCTG-3′ (SEQ ID NO. 49) and reverse primer 5′-AGCTCGCATCCGTGCATC-3′(SEQ ID NO. 49).

For expression, nucleotide sequences encoding the recombinant HLA proteins were cloned into the third-generation lentiviral expression vector, pERRL, such that their expression was under the control of an EF1α promoter. The pERRL vector was produced from the pRRLSIN.cPPT.PGK-GFP.WPRE vector (Addgene, #12252) by exchanging the human PGK promoter for the human EF1 promoter and inserting an XbaI restriction site in the 5′ multiple cloning site (MCS).

Lentivirus was produced as previously described. Briefly, 293 T cells (American Type Culture Collection) were transiently transfected with 15 μg of pERRL, 18 μg of pRSV-Rev (Addgene, #12253), 18 μg of pMDLg/pRRE (Addgene, #12251), and 7 μg of pCI-VSVG (Addgene, #1733) using Lipofectamine 2000 (Invitrogen). Supernatants were collected at 24 and 48 hours after transfection and concentrated by centrifugation at ≥30,000 g for ≥6 hours. Viral pellets were combined and resuspended in RPMI 1640 complete medium and used to transduce host cells.

Host cell lines used were K562 cells, modified K562 cells, 721.221 and BAF3 cells (K562 cells are frequently used for expanding T cells and NK cells). In some experiments modified K562 cells were used that were modified to express CD64, CD86, CD137L, truncated CD19, eGFP, and membrane-bound IL-21. These modified K562 cells referred to herein as “K562 c9.mbIL-21” cells or “K562 mod” cells.

Host cells were cultured in RPMI 1640 supplemented with 10% fetal bovine serum (FBS) and 1% penicillin-streptomycin.

Host cells were transduced with the lentiviral vectors described above containing a nucleic acid sequence encoding the given recombinant HLA protein. A K62 cell line referred to herein as “K562 c9.mbIL-21.E:G01” was generated by transducing K562 c9.mbIL-21 cells with a lentiviral vector encoding HLA-E0103:G01 (SEQ ID NO. 1) and selecting a subclone with high surface expression of HLA-E (as determined by fluorescence activated cell sorting) by limiting

For experiments involving T cells certain T cell sub-populations were isolated by flow sorting. The sorted cells were rested overnight in complete media (RPMI with Penicillin/Streptomycin, L-Glutamine, and 10% FCS) with 100U/mL IL-2, at 1 million cells per mL in a humidified incubator at 37° C. and 5% CO2. The following day, an equal number of irradiated host cells were added to the T cell culture in an equal volume of complete media. Twice a week (e.g. Monday and Friday), the T cells were counted and re-stimulated with an equal number of irradiated host cells in fresh complete media containing 100U/mL IL-2 (1 million total cells per mL, 500,000 each of T cells and host cells).

For experiments involving NK cells NK cells were isolated by flow sorting, or isolated by magnetic cell separation (MACS, Miltenyi), or examined within the total peripheral blood mononuclear call (PBMC) population. NK cells were cultured in media containing 200U/mL of IL-2.

Unless stated otherwise, the above methods of variations of these methods were employed in the studies described in the following Examples.

### Example 2

### Recombinant HLA Proteins are Successfully Expressed on the Surface of Host Cells

Numerous recombinant HLA proteins were generated and expressed in multiple cell types. Fifteen of the recombinant HLA proteins listed in Table A were expressed in 721.221 host cells, twelve were expressed in K562 host cells, and seven were expressed in BAF3 host cells.

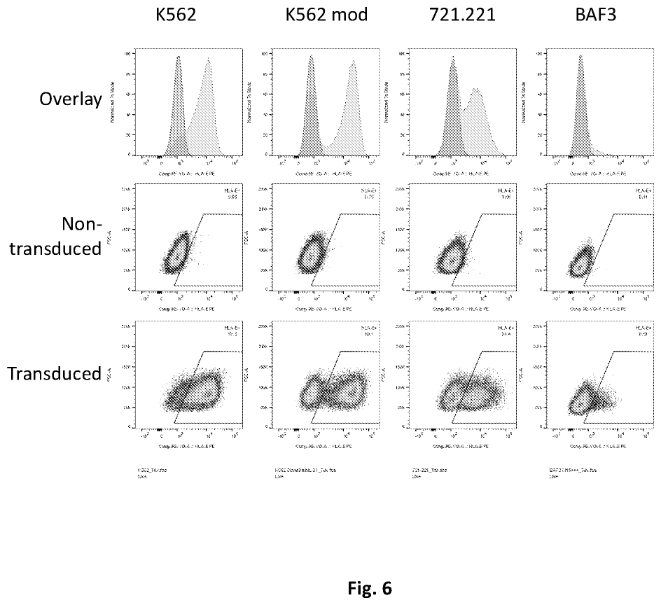

FIG. 6 provides data from flow cytometry experiments performed using four cell types—K562 cells, modified K562 cells expressing CD64, CD137L, CD86, truncated CD19, and mbIL-21 (“K562 mod”), 721.221 cells, and BAF3 cells. The cells were stained with an anti-human HLA-E antibody conjugated to a fluorochrome. The middle row shows flow cytometry scatter plot data from non-transduced cells. The bottom row shows flow cytometry scatter plot data from cells transduced with a construct containing a nucleotide sequence encoding HLA-E0103:G01 (SEQ ID NO. 1). The top row provides overlay graphs showing the data from the non-transduced (dark gray) and transduced (light gray) cell populations. This data demonstrates successful cell surface expression of the HLA-E chimera, implying successful presentation of the nonapeptide.

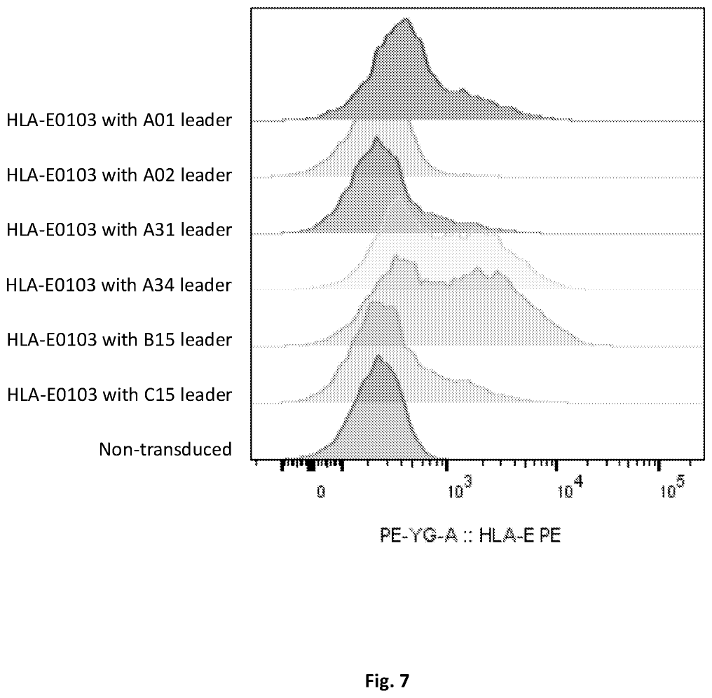

FIG. 7. provides overlaid graphs showing expression of HLA-E0103 chimeras comprising the full leader sequences of HLA-A01 (SEQ ID NO. 2), HLA-A02 (SEQ ID NO. 3), HLA-A31 (SEQ ID NO. 4), HLA-A34 (SEQ ID NO. 5), HLA-B15 (SEQ ID NO. 7), or HLA-C15 (SEQ ID NO. 15) in murine BAF3 cells expressing human beta-2 microglobulin. The bottom graph provides data from non-transduced cells.

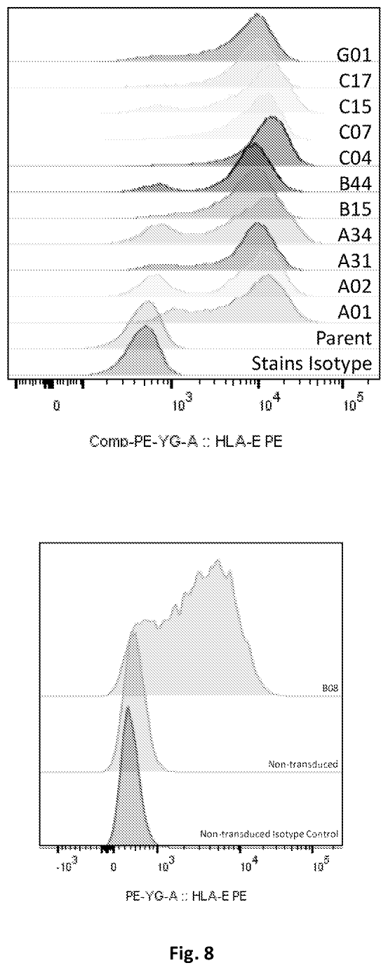

The upper panel of FIG. 8 provides overlaid graphs showing expression of HLA-E0103 chimeras comprising HLA-A01 (SEQ ID NO. 2), -A02 (SEQ ID NO. 3), -A31 (SEQ ID NO. 4), -A34 (SEQ ID NO. 5), -B44 (SEQ ID NO. 9), -C04 (SEQ ID NO. 12), -C07 (SEQ ID NO. 13), -C15 (SEQ ID NO. 14), -C17 (SEQ ID NO. 15), and -G01 (SEQ ID NO. 1) full leader sequences in K562 cells. The lower panel of FIG. 8 provides overlaid graphs showing expression of an HLA-E0103 chimera comprising the HLA-B08 full leader sequence (SEQ ID NO. 8) in K562 cells.

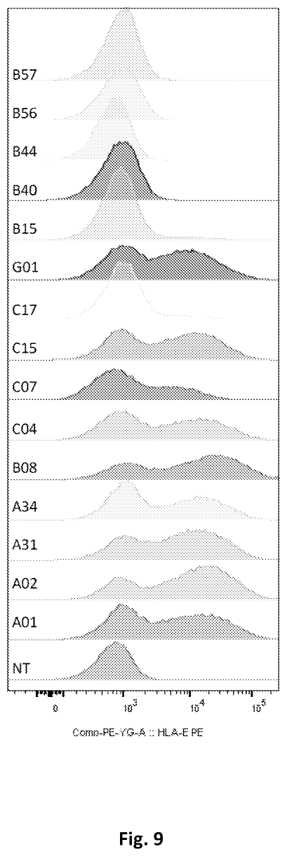

FIG. 9 provides overlaid graphs showing HLA-E expression in of 721.221 cells lines that are non-transduced or transduced with an HLA-E0103 chimera having a leader sequence from HLA-A01 (SEQ ID NO. 2), -A02 (SEQ ID NO. 3), -A31(SEQ ID NO. 4), -A34(SEQ ID NO. 5), -B08 (SEQ ID NO. 6), -C04 (SEQ ID NO. 12), -C07 (SEQ ID NO. 13), -C15 (SEQ ID NO. 14), -C17 (SEQ ID NO. 15), -G01 (SEQ ID NO. 1), -B15 (SEQ ID NO. 7), -B40 (SEQ ID NO. 8), -B44 (SEQ ID NO. 9), -B56 (SEQ ID NO. 10), or B57 (SEQ ID NO. 11).

### Example 3

Activity of Host Cells Expressing Recombinant HLA Proteins on NK Cells & T cells

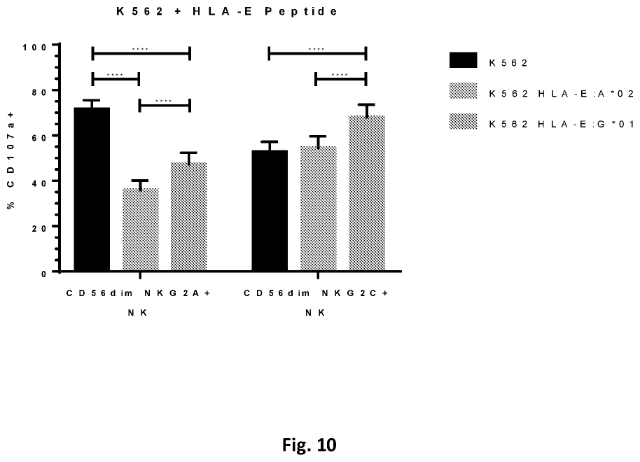

Experiments were performed to investigate the activity of the recombinant HLA proteins of the present invention on NK cells. FIG. 10. Provides data showing NKG2A+ and NKG2C+NK cell degranulation (as determined based on CD107a+ mobilization using standard assays known in the art) in response to exposure to K562 cells expressing two different HLA-E chimeras—HLA-E0103:G01 (i.e., SEQ ID NO. 1) and HLA-E0103:A02 (i.e., SEQ ID NO, 3). NK cells from 12 donors were used. These results demonstrate that K562 cells expressing the recombinant HLA molecules exhibit functional activity on NK cells, in this case exhibiting inhibitory effects on NKG2A+NK cells and activation of NKG2C+NK cells.

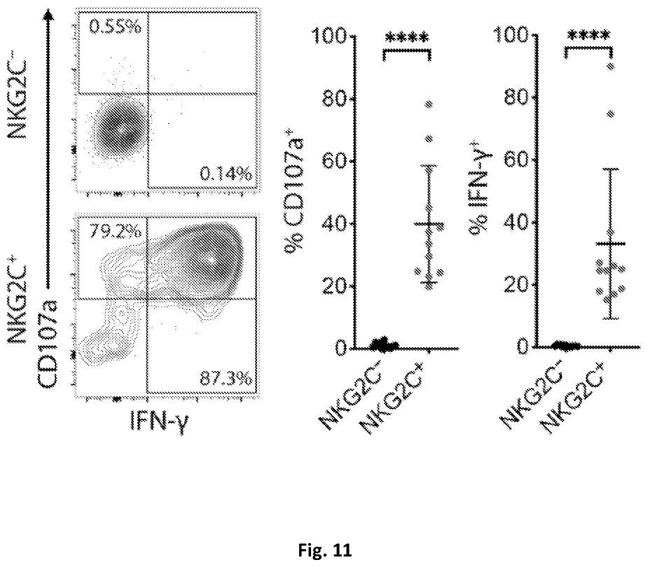

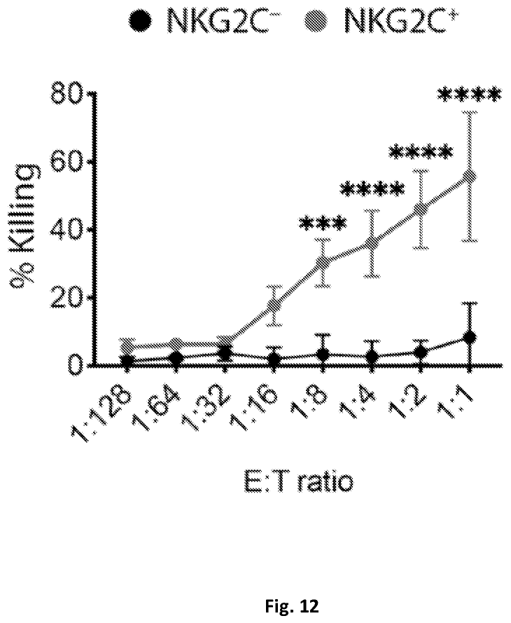

Experiments were also performed to investigate the activity of recombinant HLA proteins on T cells. FIG. 11 & FIG. 12 provide results of studies in which NKG2C+CD8+ T cells were co-incubated with K562 cells expressing HLA-E0103:G01 (i.e., SEQ ID NO. 1). The NKG2C+CD8+ T cells robustly responded, as measured by CD107 mobilization and IFN-g production, whereas the NKG2C-CD8+ T cells did not respond (FIG. 11). The NKG2C+CD8+ T cells also effectively killed the K562 cells (FIG. 12), demonstrating the ability of cells expressing the recombinant HLA proteins of the present invention to activate T cells. Additional studies demonstrating the activity of K562 cells expressing HLA-E0103:G01 are provided in Sottile et al., “Human cytomegalovirus expands a CD8+ T cell population with loss of BCL11B expression and gain of NK cell identity,” Sci. Immunol., 6, 6968 (2021), the contents of which are hereby incorporated by reference.

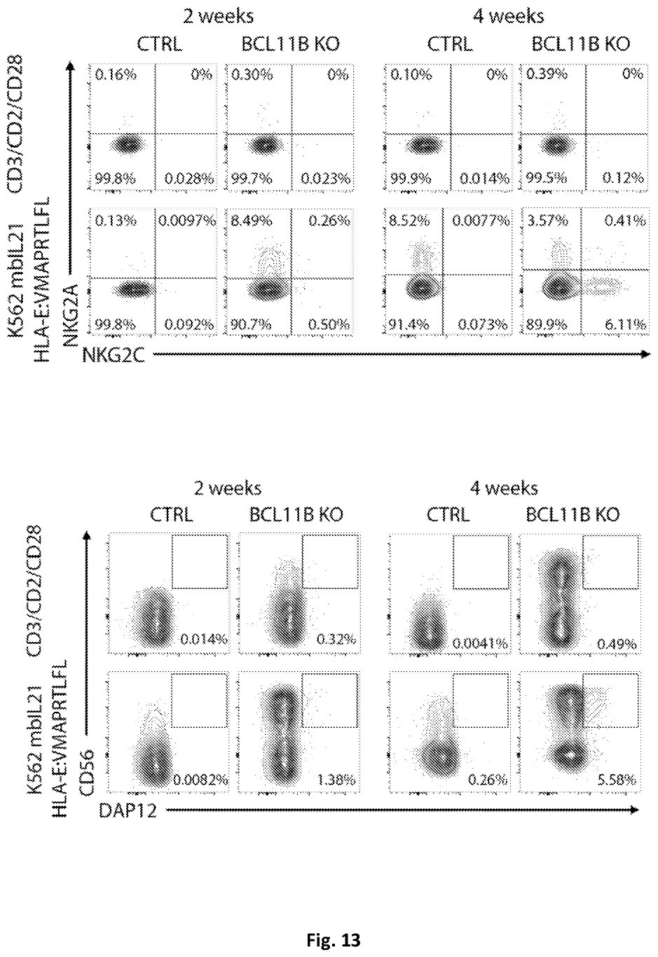

Furthermore, additional studies showed that cells expressing the recombinant HLA proteins of the present invention could induce de novo T cell populations. Specifically, when CD8+ T cells in which the BCL11B gene had been knocked out (“BCL11B KO” cells) cells were exposed to K562 cells expressing HLA-E0103: G01 for 4 weeks a de novo NKG2C+CD8+ T cell population was induced. See FIG. 13 which provides FACS plots showing the frequency of NKG2A+ versus NKG2C+ and CD56+DAP12+CD8+ T cells following culture of control and BCL11B KO cells with K562 cells expressing HLA-E0103:G01.

We claim:

1. A recombinant HLA protein comprising, from N-terminal to C-terminal: (a) a leader peptide from an HLA-A, HLA-B, HLA-C, or HLA-G molecule, and (b) an HLA-E protein, wherein the recombinant HLA protein does not comprise an HLA-E leader peptide.

2. The recombinant HLA protein of claim 1, wherein there are no intervening amino acids between the leader peptide and the HLA-E protein.

3. The recombinant HLA protein of claim 1, wherein the leader peptide comprises amino acids 3-11 of any one of SEQ ID NOs. 33-47.

4. The recombinant HLA protein of claim 1, wherein the leader peptide comprises any one of SEQ ID NOs. 33-47.

5. The recombinant HLA protein of claim 1, wherein the HLA-E protein comprises amino acids 22-358 of SEQ ID NO. 31 or SEQ ID NO. 32.

6. The recombinant HLA protein of claim 1, comprising any one of SEQ ID NO. 1 to SEQ ID NO. 30.

7. The recombinant HLA protein of claim 1, consisting of any one of SEQ ID NO. 1 to SEQ ID NO. 30.

8. A recombinant HLA protein produced intracellularly from a recombinant HLA protein of any of claims 1-7.

9. The recombinant HLA protein of claim 8, wherein the protein is produced intracellularly by proteolytic removal of the leader peptide by a signal peptidase.

10. A nucleic acid molecule encoding a recombinant HLA protein according to any of the preceding claims.

11. The nucleic acid molecule of claim 10, wherein the nucleic acid molecule is a DNA molecule.

12. The nucleic acid molecule of claim 10, wherein the nucleic acid molecule is an RNA molecule.

13. The nucleic acid molecule according to claim 11, wherein the nucleic acid molecule encoding the recombinant HLA protein is operatively linked to a promoter.

14. The nucleic acid molecule according to claim 13, wherein promoter is an EF1α promoter.

15. The nucleic acid molecule according to any of claims 10-14, wherein the nucleic acid molecule is codon optimized for human expression.

16. A vector comprising a nucleic acid molecule according to any of claims 10-15.

17. The vector according to claim 16, wherein the vector is a lentiviral vector.

18. A host cell comprising a recombinant HLA protein according to any of claims 1-9.

19. A host cell comprising a nucleic acid molecule according to any of claims 10-15 or a vector according to any of claims 16-17.

20. A host cell according to claim 18 or claim 19, wherein the cell is a 721.221 cell.

21. A host cell according to claim 18 or claim 19, wherein the cell is a K562 cell.

22. A host cell according to claim 18 or claim 19, wherein the cell is a K562 c9.mbIL-21 cell.

23. A host cell according to claim 18 or claim 19, wherein the cell is a BAF3 cell.

24. A composition comprising a recombinant HLA protein according to any of claims 1-9, a nucleic acid molecule according to any of claims 10-15, a vector according to any of claims 16-17, or a host cell according to any of claims 18-23.

25. A method for activating or expanding T cells or NK cells, the method comprising contacting T cells or NK cells with host cells according to any of claims 18-23.

26. The method of claim 25, wherein the T cells are CD8+ T cells.

27. The method of claim 26, wherein the T cells are NKG2C+CD8+ T cells.

28. The method of any of claims 25-27, wherein the contacting is in vivo.

29. The method of any of claims 25-27, wherein the contacting is in vitro.

30. The method of claim 29, wherein the contacting comprises culturing the host cells and the T cells or NK cells in the presence of a suitable culture medium.

31. The method of claim 30, wherein the culture medium comprises IL-2.
